SEQUENCE LISTING (17 sequences)

SEQ ID NO: 1 - protein, 23 aa, Xenopus laevis
GIGKFLHSAKKFGKAFVGEIMNS

SEQ ID NO: 2 - DNA, 69 bp, Xenopus laevis
ggcattggtaaatttttgcactcagcaaaaaaatttggcaaagcttttgtgggcgagattatgaattca

SEQ ID NO: 3 - protein, 18 aa, Sus scrofa
RGGGLCYCRRRFCVCVGR

SEQ ID NO: 4 - protein, 149 aa, Sus scrofa
METQRASLCLGRWSLWLLLLALVVPSASAQALSYREAVLRAVDRLNEQSSEANLYRLLELDQPPKADEDPGTPKPVSFTVKETVCPRPTRQPPELCDFKENGRVKQCVGTVTLDQIKDPLDITCNEVQGVRGGRLCYCRRRFCVCVGRG

SEQ ID NO: 5 - protein, 70 aa, Apis mellifera
MKFLVNVALVFMVVYISYIYAAPEPEPAPEPEAEADAEADPEAGIGAVLKVLTTGLPALISWIKRKRQQG

SEQ ID NO: 6 - protein, 170 aa, Homo sapiens
MKTQRDGHSLGRWSLVLLLLGLVMPLAIIAQVLSYKEAVLRAIDGINQRSSDANLYRLLDLDPRPTMDGDPDTPKPVSFTVKETVCPRTTQQSPEDCDFKKDGLVKRCMGTVTLNQARGSFDISCDKDNKRFALLGDFFRKSKEKIGKEFKRIVQRIKDFLRNLVPRTES

SEQ ID NO: 7 - protein, 29 aa, Phyllomedusa oreades
GLWSTIKQKGKEAAIAAAKAAGQAALGAL

SEQ ID NO: 8 - protein, 39 aa, Glossina morsitans
GWLKKIGKKIERVGQNTRDATVKGLEVAQQAANVAATVR

SEQ ID NO: 9 - protein, 26 aa, Litoria gilleni
GLLSVLLGSVAKHVLPHVVPVIAEHL

SEQ ID NO: 10 - protein, 18 aa, artificial sequence
  synthetic anti-microbial peptide ovispirin
XNLRRIIRKIIHIIKKYG

SEQ ID NO: 11 - protein, 21 aa, Trichoderma viride
  MISC_FEATURE (1)..(2): X = any amino acid
XXPXAXAQXVXGLXPVXXEQX

SEQ ID NO: 12 - DNA, 83 bp, artificial sequence
  mutated Magainin II sequence
gatcctggggcattggtaaatttttgcactcagcaaaaaaatttggcaaagcttttgtgggcgagattatgaattcataatag

SEQ ID NO: 13 - DNA, 83 bp, artificial sequence
  mutated Magainin II sequence
tcgactattatgaattcataatctcgcccacaaaagctttgccaaatttttttgctgagtgcaaaaatttaccaatgccccag

SEQ ID NO: 14 - DNA, 24 bp, artificial sequence
  primer sequence
ggtcgtcagactgtcgatgaagcc

SEQ ID NO: 15 - protein, 6 aa, Artificial Sequence
  Weak groupd of conserved served residues
SNDEQK

SEQ ID NO: 16 - protein, 6 aa, Artificial Sequence
  Weak groupd of conserved served residues
NDEQHK

SEQ ID NO: 17 - protein, 6 aa, Artificial Sequence
  Weak groupd of conserved served residues
NEQHRK

(19) United States
(12) Patent Application Publication
(10) Pub. No.: US 2011/0009291 A1
(43) Pub. Date: Jan. 13, 2011

(54) METHODS AND COMPOSITIONS FOR INCREASING MEMBRANE PERMEABILITY

(75) Inventors: Rachel R. Chen, Marietta, GA (US); Xuan Guo, Suwanee, GA (US)

Correspondence Address: Pabst Patent Group LLP, 1545 PEACHTREE STREET NE, SUITE 320, ATLANTA, GA, 30309, US

(73) Assignees: Georgia Tech Research Corporation; Virginia Commonwealth University

(21) Appl. No.: 12/846,646

(22) Filed: Jul. 29, 2010

Related U.S. Application Data
(62) Division of application No. 11/813,980, filed on Jul. 13, 2007.
(60) Provisional application No. 60/644,476, filed on Jan. 16, 2005.

(51) Int. Cl. C40B 40/02; C12P 21/06; A62D 3/02; C12N 15/74

(52) U.S. Cl. 506/14; 435/69.1; 435/262.5; 435/471

(57) ABSTRACT

Methods and compositions for increasing membrane permeability are provided. One aspect provides protein resulting from a fusion between a membrane-active peptide and second peptide. Nucleic acids and vectors encoding the pore forming fusion proteins are also provided.

This application is being filed on 17 Jan. 2006, as a PCT International Patent application in the name of Georgia Tech Research Corporation, a U.S. national corporation, and Rachel R. Chen a citizen of the U.S., and Xuan Guo, a citizen of China and claims priority to and benefit of U.S. Provisional Patent Application No. 60/644,476 filed on Jan. 16, 2005, and where permissible, is incorporated by reference in its entirety.

### STATEMENT REGARDING FEDERALLY SPONSORED RESEARCH OR DEVELOPMENT

Aspects of the work described herein were supported, in part, under Grant No. BES0455194 awarded by the National Science Foundation's Technology for Sustainable Environment (TSE) Program. The US government may have certain rights in the disclosed subject matter.

### BACKGROUND

1. Technical Field

The disclosed subject matter is generally directed to the field of biotechnology, in particular to recombinant cells expressing membrane permeablizing peptides and methods of their use.

2. Related Art

A wide variety of commercially important commodity or specialty chemicals are produced through metabolic engineering, the manipulation of living organisms to achieve desirable metabolic substrates, products and/or byproducts. A fundamental issue in all biotechnology processes concerns the flow of molecules into and from cells. Substrates or nutrients must be transported into cells and reach intracellular catalyst sites, for example intracellular enzymes, at a sufficiently high rate to ensure high reaction rates and thereby productivity. Similarly, once the products are made inside the cell, the products need to be transported to the desired location, preferably extracellularly, to facilitate recovery. Unfortunately, the metabolic network inside various cell types used in biotechnology is not optimized for maximal production of a metabolite useful for human exploitation. Similarly, cellular membrane systems of these cells are not optimized for maximal uptake of a substrate or for maximal secretion of a desired substance. Because the transport of a substrate or a product is often the rate-limiting step of an overall bioprocess, cell systems having increased membrane permeability to specific reagents or products would enable recovery of larger amounts of product in shorter time periods compared to existing systems.

### SUMMARY

Aspects of the present disclosure provide methods and compositions for increasing membrane permeability. One aspect provides a fusion protein between a membrane-active peptide, for example an antimicrobial peptide, and a second peptide. Another aspect provides a nucleic acid and vectors encoding the disclosed fusion proteins.

Still another aspect provides a cell, for example a bacterium, that expresses a membrane-active peptide or a fusion protein thereof.

Another aspect provides a method for increasing the permeability of a membrane of cell by contacting the membrane with one or more of the disclosed membrane-active peptides or fusion proteins thereof. The membrane-active peptide or fusion protein thereof can be used in combination with additional permeabilizing agents, including, but not limited to, toxins, surfactants, detergents, organic solvents, or freeze thaw techniques.

Another aspect provides a method for increasing the rate and yield of an enzymatic process from a cell compared to a control. In this method, the cell expresses a nucleic acid, for example a gene encoding one or more of the disclosed membrane-active peptides or fusion proteins thereof. The membrane-active peptides or fusion proteins thereof increases membrane permeability of the cell allowing extracellular reactants to translocate an outer membrane of the cell and contact an enzyme located within the cell. Reactants can enter the cell at a higher rate compared to a control cell as a result of the increase in outer membrane permeability. Therefore, the enzyme within the cell can produce products at a higher rate, resulting in higher productivity, higher product concentrations, and higher yield. In some embodiments, the cell is a prokaryotic cell or eukaryotic cell. In some embodiments, the cell is a gram negative bacterium, gram positive bacterium, a yeast, an insect cell, or a mammalian cell

Another aspect provides a method for bioremediation. In this a method a cell containing an enzyme that converts or is capable of converting a toxic reactant into a non-toxic product is contacted with one or more of the disclosed membrane-active peptides or fusion proteins thereof, optionally in combination with at least one additional membrane permeabilizing agent. Alternatively, the cell is transfected to express one or more of the disclosed membrane-active peptides or fusion proteins thereof. The cell is then contacted with the toxic reactant under conditions that favor the conversion of the toxic reactant into a non-toxic product.

Another embodiment provides a method for controlling membrane permeability of a cell by expressing one or more nucleic acids, for example a gene encoding one or more of the disclosed membrane-active peptides or fusion proteins thereof. Membrane permeability increases with an increase in number of nucleic acids expressing the membrane-active peptides or fusion proteins in the cell. Alternatively, membrane permeability can increase in response to an increase in the amount of inducer from an inducible promoter operably linked to the nucleic acids. Lastly, promoters of different strengths can be used so that the amount of membrane-active peptides or fusion proteins expressed by the cell is controlled. Higher levels of expression of the disclosed membrane-active peptides or fusion proteins correspond to higher membrane permeability increases compared to low levels of expression of the membrane-active peptides or fusion proteins. Alternatively, the cell can be engineered to express a predetermined amount of the disclosed membrane-active peptides or fusion proteins and various amounts of a permeabilizing agent, including the disclosed fusion proteins can be added to the cell to increase membrane permeability to a desired level.

Another aspect provides a cellular array comprising a cell expressing one or more of the disclosed membrane-active peptides or fusion proteins.

Yet another aspect provides a kit containing one or more of the membrane-active peptides or fusion proteins or a cell expressing one or more of the disclosed proteins.

### BRIEF DESCRIPTION OF THE FIGURES

The drawings form part of the present specification and are included to further demonstrate certain aspects of the present invention. The invention may be better understood by reference to one or more of these drawings in combination with the detailed description of specific embodiments presented herein.

FIG. IA shows the sequence of the Magainin II gene.

FIG. IB shows a construction of an exemplary membrane permeabilizing polypeptide, Magll-MalE-fusion protein. The underlined bases were optimized according to the E. coli codon usage.

FIG. 2 shows SDS-PAGE gel (A) and Western blot (B) analysis of expression of Magll-MalE fusion protein in E609/cM13. The samples were loaded in the following order: Protein standard markers (lane M), MaIE standard (42.5 kDa, lane 1), E609/cM13 induced w/O 0.1 mM IPTG at 3 hr (lane 2), E609/cM13 induced w/O.1 mM IPTG at 3 hr (lane 3), E609/cM13 induced w/0.3 mM IPTG at 3 hr (lane 4), E609/cM13 induced w/0.5 mM IPTG at 3 hr (lane 5), E609/cM13 induced w/O mM IPTG at 4 hr (lane 6), E609/cM13 induced w/0.1 mM IPTG at 4 hr (lane 7), E609/cM13 induced w/0.3 mM EPTG at 4 hr (lane 8), E609/cM13 induced w/0.5 mM IPTG at 4 hr (lane 9), E609/cM13 induced w/O mM EPTG at 5 hr (lane 10), E609/cM13 induced w/O 0.1 mM EPTG at 5 hr (lane 11), E609/cM13 induced w/0.3 mM IPTG at 5 hr (lane 12), E609/cM13 induced w/0.5 mM IPTG at 5 hr (lane 13).

FIG. 3 shows a graph indicating NPN uptake factors for E609/c2x and E609/cM13 at different sampling time points.

FIG. 4 shows extracellular β-lactamase (Nitrocefm) activities from cell culture of E609 E609/c2x and E609/cM13 at different EPTG concentrations

FIG. 5 shows a graph indicating whole-cell β-glucuronidase activities with E609/c2x and E609/cM13 at different sampling time points.

FIG. 6 shows SDS-PAGE (A) and Western blot (B) analysis of locations of the expressed Magl1-MalE fusion protein in E609/cM13 induced with 0.1 mM EPTG. The samples were taken at 3 hr. Protein standard marker (lane M)₅MaIE standard (42.5 kDa, lane 1), whole cell fraction 10 μl (lane 2), concentrated periplasmic fraction 50 μl (lane 3), and concentrated extracellular (supernatant) fraction 50 μl (lane 4).

### DETAILED DESCRIPTION

### Definitions

Unless otherwise noted, technical terms are used according to conventional usage. Definitions of common terms in molecular biology may be found in Lewin, Genes VII, published by Oxford University Press, 2000; Kendrew et al. (eds.), The Encyclopedia of Molecular Biology, published by Wiley-Interscience., 1999; and Robert A. Meyers (ed.), Molecular Biology and Biotechnology, a Comprehensive Desk Reference, published by VCH Publishers, Inc., 1995; Ausubel et al. (1987) Current Protocols in Molecular Biology, Green Publishing; Sambrook and Russell. (2001) Molecular Cloning: A Laboratory Manual 3rd. edition.

In order to facilitate understanding of the disclosure, the following definitions are provided:

An “antimicrobial peptide” refers to oligo- or polypeptides that kill microorganisms or inhibit their growth including peptides that result from the cleavage of larger proteins or peptides that are synthesized ribosomally or non-ribosomally. Generally, antimicrobial peptides are cationic molecules with spatially separated hydrophobic and charged regions. Exemplary antimicrobial peptides include linear peptides that form an α-helical structure in membranes or peptides that form/3-sheet structures optionally stabilized with disulfide bridges in membranes. Representative antimicrobial peptides include, but are not limited to cathelicidins, defensins, dermcidin, and more specifically magainin 2, protegrin, protegrin-1, melittin, 11-37, dermaseptin 01, cecropin, caerin, ovispirin, and alamethicin. It will be appreciated that antimicrobial peptides include peptides from vertebrates and non-vertebrates, including plants, humans, fungi, microbes, and insects. Antimicrobial peptides include those peptides that increase membrane permeability, for example by forming a pore in the membrane.

An “array”, unless a contrary intention appears, includes any one-, two- or three-dimensional arrangement of addressable regions each having at least one unit of cells optionally in combination with a particular chemical moiety or moieties (for example, biopolymers, antibodies, reactants) associated with that region. An array is “addressable” in that it has multiple regions of different moieties (for example, different cell types or chemicals) such that a region (a “feature” or “spot” of the array) at a particular predetermined location (an “address”) on the array will detect a particular target or class of targets (although a feature may incidentally detect non-targets of that feature). Array features are typically, but need not be, separated by intervening spaces.

An “array layout” refers to one or more characteristics of the array or the features on it. Such characteristics include one or more of: feature positioning on the substrate; one or more feature dimensions; some indication of an identity or function (for example, chemical or biological) of a moiety at a given location; how the array should be handled (for example, conditions under which the array is exposed to a sample, or array reading specifications or controls following sample exposure).

A “pulse jet” is a device which can dispense drops in the formation of an array. Pulse jets operate by delivering a pulse of pressure to liquid adjacent to an outlet or orifice such that a drop will be dispensed therefrom (for example, by a piezoelectric or thermoelectric element positioned in a same chamber as the orifice).

When referring to expression, “control sequences” means DNA sequences necessary for the expression of an operably linked coding sequence in a particular host organism. Control elements may be positive or negative control elements. Positive control elements require binding of a regulatory element for initiation of transcription. Many such positive and negative control elements are known. A negative control element is one that is removed for activation. Many such negative control elements are known, for example operator/repressor systems. For example, binding of IPTG to the lac repressor dissociates from the lac operator to activate and permit transcription. Other negative elements include the E. coli tip and lambda systems.

Control sequences that are suitable for prokaryotes, for example, include a promoter, optionally an operator sequence, a ribosome binding site, and the like. The term “promoter” refers to a regulatory nucleic acid sequence, typically located upstream (5^{!}) of a gene or protein coding sequence that, in conjunction with various elements, is responsible for regulating the expression of the gene or protein coding sequence. Eukaryotic cells are known to utilize promoters, polyadenylation signals, and enhancers. Where heterologous control elements are added to promoters to alter promoter activity as described herein, they are positioned within or adjacent the promoter sequence so as to aid the promoter's regulated activity in expressing an operationally linked polynucleotide sequence. Examples of control or regulatory elements include, but are not limited to, a TATA box, operators, enhancers, and the like.

The term “cell” refers to a membrane-bound biological unit capable of replication or division.

The term “construct” refers to a recombinant genetic molecule comprising one or more isolated polynucleotide sequences of the invention.

Genetic constructs used for transgene expression in a host organism comprise in the 5′-3′ direction, a promoter sequence; a sequence encoding a microbial peptide disclosed herein; and a termination sequence. The construct may also comprise selectable marker gene(s) and other regulatory elements for expression.

A “chamber” references an enclosed volume (although a chamber may be accessible through one or more ports).

A “control” refers to a sample of material which is known to be substantially similar to a sample containing the disclosed fusion protein, except that the control sample may not contain or express the fusion protein.

The term “heterologous” refers to elements occurring where they are not normally found. For example, a promoter may be linked to a heterologous nucleic acid sequence, e.g., a sequence that is not normally found operably linked to the promoter. When used herein to describe a promoter element, heterologous means a promoter element that differs from that normally found in the native promoter, either in sequence, species, or number. For example, a heterologous control element in a promoter sequence may be a control/regulatory element of a different promoter added to enhance promoter control, or an additional control element of the same promoter. Heterologous peptide refers to a peptide that is not found in the host organism.

As used herein, the term “homologues” is generic to “orthologues” and “paralogies”. The term “orthologues” refers to separate occurrences of the same gene in multiple species. The separate occurrences have similar, albeit nonidentical, amino acid sequences, the degree of sequence similarity depending, in part, upon the evolutionary distance of the species from a common ancestor having the same gene. As used herein, the term “paralogues” indicates separate occurrences of a gene in one species. The separate occurrences have similar, albeit nonidentical, amino acid sequences, the degree of sequence similarity depending, in part, upon the evolutionary distance from the gene duplication event giving rise to the separate occurrences.

As used herein, the phrase “induce expression” means to increase the amount or rate of transcription and/or translation from specific genes by exposure of the cells containing such genes to an effector or inducer reagent or condition.

An “inducer” is a chemical or physical agent which, when applied to a population of cells, will increase the amount of transcription from specific genes. These are usually small molecules whose effects are specific to particular operons or groups of genes, and can include sugars, phosphate, alcohol, metal ions, hormones, heat, cold, and the like. For example, isopropyl (beta)-D-thiogalactopyranoside (IPTG) and lactose are inducers of the tacit promoter, and L-arabinose is a suitable inducer of the arabinose promoter.

“PTG” is the compound “isopropyl (beta)-D-thiogalactopyranoside”, and is used herein as an inducer of lac operon. IPTG binds to a lac repressor effecting a conformational change in the lac repressor that results in dissociation of the lac repressor from the lac operator. With the lac repressor unbound, an operably linked promoter is activated and downstream genes are transcribed.

As used herein, the term “mammal” refers to any animal classified as a mammal, including humans, domestic and farm animals, and zoo, sports, or pet animals, such as dogs, horses, cats, cows, etc. The mammal can be, for example, human.

By the terms “amino acid residue” and “peptide residue” is meant an amino acid or peptide molecule without the —OH of its carboxyl group (C-terminally linked) or the proton of its amino group (N-terminally linked). In general the abbreviations used herein for designating the amino acids and the protective groups are based on recommendations of the IUPAC-IUB Commission on Biochemical Nomenclature (see Biochemistry (1972) 11:1726-1732). Amino acid residues in peptides are abbreviated as follows: Alanine is Ala or A; Cysteine is Cys or C; Aspartic Acid is Asp or D; Glutamic Acid is GIu or E; Phenylalanine is Phe or F; Glycine is GIy or G; Histidine is His or H; Isoleucine is He or 1; Lysine is Lys or K; Leucine is Leu or L; Methionine is Met or M; Asparagine is Asn or N; Proline is Pro or P; Glutamine is GIn or Q; Arginine is Arg or R; Serine is Ser or S; Threonine is Thr or T; Valine is VaI or V; Tryptophan is Trp or W; and Tyrosine is Tyr or Y. Formylmethionine is abbreviated as fMet or fM. By the term “residue” is meant a radical derived from the corresponding α-amino acid by eliminating the OH portion of the carboxyl group and the H portion of the α-amino group. The term “amino acid side chain” is that part of an amino acid exclusive of the —CH(NH₂)COOH portion, as defined by K. D. Kopple, “Peptides and Amino Acids”, W. A. Benjamin Inc., New York and Amsterdam, 1966, pages 2 and 33; examples of such side chains of the common amino acids are —CH₂CH₂SCH₃ (the side chain of methionine), —CH₂(CH₃)—CH₂CH₃ (the side chain of isoleucine), —CH₂CH(CH₃)₂ (the side chain of leucine) or —H (the side chain of glycine).

A peptide is “operably linked” when it is placed into a functional relationship with another peptide, polypeptide or protein. For example, an antimicrobial peptide is operably liked to a second peptide so that both parts of the fusion protein retain a biological function.

A “region” refers to any finite small area on the array that can be illuminated and any resulting fluorescence therefrom simultaneously (or shortly thereafter) detected, for example a pixel.

“Plasmids” are designated by a lower case “p” preceded and/or followed by capital letters and/or numbers. The starting plasmids herein are either commercially available, publicly available on an unrestricted basis, or can be constructed from available plasmids in accord with published procedures. In addition, equivalent plasmids to those described are known in the art and will be apparent to the ordinarily skilled artisan.

As used herein, “polypeptide” refers generally to peptides and proteins having more than about ten amino acids. The polypeptides can be “exogenous,” meaning that they are “heterologous,” i.e., foreign to the host cell being utilized, such as human polypeptide produced by a bacterial cell. Exogenous also refers to substances that are added from outside cells, not endogenous (produced by cells).

It will also be appreciated that throughout the present application, that words such as “top”, “upper”, and “lower” are used in a relative sense only.

When one item is indicated as being “remote” from another, this is referenced that the two items are at least in different buildings, and may be at least one mile, ten miles, or at least one hundred miles apart. “Communicating” information references transmitting the data representing that information as electrical signals over a suitable communication channel (for example, a private or public network). “Forwarding” an item refers to any means of getting that item from one location to the next, whether by physically transporting that item or otherwise (where that is possible) and includes, at least in the case of data, physically transporting a medium carrying the data or communicating the data.

Reference to a singular item, includes the possibility that there are plural of the same items present.

“Transformed,” “transgenic,” “transfected” and “recombinant” refer to a host organism such as a bacterium or eukaryotic cell into which a heterologous nucleic acid molecule has been introduced. The nucleic acid molecule can be stably integrated into the genome of the host or the nucleic acid molecule can also be present as an extrachromosomal molecule. Such an extrachromosomal molecule can be auto-replicating. Transformed cells are understood to encompass not only the end product of a transformation process, but also transgenic progeny thereof. A “non-transformed,” “non-transgenic,” or “non-recombinant” host refers to a wild-type organism, e.g., a bacterium or eukaryotic cell, which does not contain the heterologous nucleic acid molecule.

A “transformed cell” refers to a cell into which has been introduced a nucleic acid molecule, for example by molecular biology techniques. As used herein, the term transformation encompasses all techniques by which a nucleic acid molecule might be introduced into such a cell, plant or animal cell, including transfection with viral vectors, transformation by Agrobacterium, with plasmid vectors, and introduction of naked DNA by electroporation, lipofection, and particle gun acceleration and includes transient as well as stable transformants.

The term “membrane-active peptide” refers to a peptide capable of insertion into a lipid membrane, typically a bilayer lipid membrane. Membrane-active peptide is generic to antimicrobial peptide and includes those peptides that may not kill microorganisms but nonetheless insert or associate with a membrane and increase the permeability of the membrane.

The term “vector” refers to a nucleic acid molecule which is used to introduce a polynucleotide sequence into a host cell, thereby producing a transformed host cell. A “vector” may comprise genetic material in addition to the above-described genetic construct, e.g., one or more nucleic acid sequences that permit it to replicate in one or more host cells, such as origin(s) of replication, selectable marker genes and other genetic elements known in the art (e.g., sequences for integrating the genetic material into the genome of the host cell, and so on).

Methods recited herein may be carried out in any order of the recited events which is logically possible, as well as the recited order of events.

All patents and other references cited in this application, are incorporated into this application by reference where permissible except insofar as they may conflict with those of the present application (in which case the present application prevails).

### Exemplary Embodiments

Increasing membrane permeability of cells can increase the production by those cells of a desired product, for example an enzymatic product or a protein produced by the cell compared to control cells. It has been discovered that expressing a membrane-active peptide, for example an antimicrobial peptide, in a cell wherein the membrane-active peptide is active on the membranes of the cell expressing the membrane-active peptide can increase membrane permeability of the cell without causing the cell to die. Expression of the membrane-active peptide can be regulated so that the cell continues to grow and divide. In certain embodiments, cells expressing the membrane-active peptide have an increased doubling time compared to control cells. Although membrane-active peptides have previously been cloned, isolated from inclusion bodies, and activated in vitro after isolation, it is believed that present disclosure represents the first time cells have been engineered to express a membrane-active peptide that is active in vivo, (i.e., on the cell expressing the membrane-active peptide), for purposeful manipulations of membrane permeability for biotechnological applications.

Accordingly, one embodiment provides a method for increasing or decreasing membrane permeability of cell comprising increasing or decreasing expression of membrane-active peptide, optionally fused to a second peptide for example a heterologous peptide, in the cell, wherein the degree of membrane permeability of the cell is correlated to the amount of membrane-active peptide expressed in the cell. High levels of membrane-active peptide expression correlated to high levels of membrane permeability, and low levels of membrane-active peptide expression correlate to low levels of membrane-permeability. Levels of membrane-permeability can be calibrated against control cells that do not express the membrane-active peptide. The cell can optionally be engineered to express a second recombinant protein. The second recombinant protein can be an enzyme, antibody, antibody fragment, peptide hormone, growth factor, insulin, cytokine, or other therapeutic polypeptide. Exemplary enzymes include those that produce specific optical isomers of chiral compounds; produce alcohols, ketones, etc.; or reduce or oxidize toxic compounds to less toxic or non-toxic compounds.

Another embodiment provides a cell comprising a first nucleic acid encoding a membrane-active peptide and a second nucleic acid encoding a second polypeptide, wherein expression of the first nucleic acid increases membrane permeability with regard to the second polypeptide, and wherein membrane permeability is controlled by controlling the expression of the first nucleic acid. The second polypeptide can be a enzyme, antibody, antibody fragment, peptide hormone, growth factor, insulin, cytokine, or other therapeutic polypeptide. Membrane permeability can be increased by increasing the expression of the first nucleic acid or decreased by decreasing the expression of the first nucleic acid. Alternatively, membrane permeability can be controlled by fusing the membrane-active peptide with a second peptide, for example a heterologous peptide. Expression of the membrane-active peptide can be controlled using methods known in the art including but not limited to operably linking the nucleic acid encoding the membrane-active peptide to an inducible promoter, strong or weak promoter, regulating vector copy number per cell, etc.

In one embodiment, the second peptide can decrease the pore forming ability of the membrane-active peptide. Without wishing to be bound by one theory, it is believed that the heterologous peptide sterically hinders the membrane-active peptide, and thereby reduces the ability of the membrane-active peptide to increase membrane permeability.

In certain embodiments, expression of the fusion protein between a membrane-active peptide and a second peptide, for example a heterologous peptide in the cell results in a lower level of membrane permeability compared to expression of the membrane-active peptide alone. The heterologous peptide can be selected based on size or ability to target the fusion protein to a specific type of membrane or area of a membrane or cell.

Antimicrobial peptides are known in the art (Biesswenger et al. (2005) Current Protein and Peptide Science, 6, 255-264). Indeed, to date over seven hundred antimicrobial peptides have been described. Antimicrobial peptides exist widely from bacteria to mammals. They are encoded by the genome and produced through regular processes of gene transcription. In addition to antibacterial effects, some peptides also have an effect on bacteria, fungi, viruses, and/or even cancer cells. It is believed that these cationic peptides interact directly with biological membranes without the need of a specific receptor. Although the mechanism of how these peptides kill cells is not clearly understood, antimicrobial peptides are considered to be promising alternative to overcome the growing antibiotic resistance problems.

In some embodiments, the antimicrobial peptide is naturally occurring and in other embodiments, variants of the antimicrobial peptides can be made. In some embodiments, the antimicrobial peptide is active against a wide variety of microbes including fungi, gram positive bacteria, and gram negative bacteria. In other embodiments, an antimicrobial peptide may be selected that is more specific for gram positive or gram negative bacteria. Antimicrobial peptides that may be utilized in the methods of the invention include cecropins, cathelicins, dermaseptins, defensins, histatins, and surfactant protein B. The antimicrobial peptide may be obtained from any species or may be synthetically or recombinantly produced.

Without wishing to be bound by one theory, it is believed that the membrane-active peptides and their corresponding fusion proteins provided herein increase membrane permeability of cell by forming a pore in the membrane or by disrupting the membrane. The pore may be formed by one membrane-active peptide or fusion protein or by a several membrane-active peptides or fusion proteins combining to form a multimeric complex. For example, one membrane-active peptide or fusion protein can produce an α-helical structure to form a pore. Alternatively, the fusion protein can adopt a/3-pleated structure in the membrane and thereby increases membrane permeability. The pore can be of sufficient size to allow small organic molecules or proteins to translocate across the membrane.

In certain embodiments, pores formed by the disclosed membrane-active peptides have an interior diameter of about 1 nm to about 7 nm. It will be appreciated that different membrane-active peptides can produce pores having different interior diameters. To increase membrane permeability to a specific compound, a membrane-active peptide that will produce pores having an interior diameter that will accommodate the compound can be used.

One embodiment provides membrane-active peptides comprising about 10 to about 200 amino acid residues, typically less than about 50 residues with net positive charges under physiological conditions. Membrane-active peptides tend to adopt different conformations depending on the environmental conditions. Many antimicrobial peptides are disordered in water, but become ordered when attached to membranes or membrane-mimicking micelles. Suitable antimicrobial peptides include (1) those that form a helical structure including alpha helix and 3,10 helix; (2) those that form a beta structure with disulfide bonds; (3) those that form beta structures without disulfide bonds (i.e.; Beta strand); (4) those that form both alpha and beta structures; (5) those that are rich in unusual amino-acid residues such as GIy, Trp or Pro; and (6) those produced by vertebrates, non-vertebrates, plants, fungi, or microbes.

Non-limiting examples of antimicrobial peptides that can be used to generate the disclosed fusion proteins include those provided in Table 1.

TABLE 1
Exemplary Antimicrobial Peptides

| | | Number of Amino |
|---|---|---|
| Peptide | Accession Number | Acids |
| Magainin 2 | gi: 14719517 (SEQ ID NO: 1) | 23 |
| Protegrin | gi: 404379 (SEQ ID NO: 3) | 18 |
| Protegrin-1 | gi: 887643 (SEQ ID NO: 4) | 149 |
| Melittin | gi: 58585154 (SEQ ID NO: 5) | 70 |
| LL-37 | gi: 1706745 (SEQ ID NO: 6) | 170 |
| Dermaseptin 01 * | gi: 41016983 (SEQ ID NO: 7) | 29 |
| cecropin | gi: 25089845 SEQ ID NO: 8) | 39 |
| Caerin | gi: 738227 (SEQ ID NO: 9) | 26 |
| Ovispirin | gi: 20663798 (SEQ ID NO: 10) | 18 |
| Alamethicin | gi: 229677 (SEQ ID NO: 11) | 21 |

Other embodiments provide variants or homologues of these antimicrobial or membrane-active peptides or fusion proteins thereof. Variants include antimicrobial peptides having at least one amino acid substituted with another. The substitution may or may not affect the function of the antimicrobial peptide, the second peptide, or the fusion protein. For example, the amino acid sequence for magainin 2 can be varied to increase or decrease the overall positive charge of the peptide. Other amino acid substitutions can be chosen to increase or decrease the hydrophobicity of the peptide. Substituted amino acids may be fully conserved “strong” residues or fully conserved “weak” residues. The “strong” group of conserved amino acid residues may be any one of the following groups: STA, NEQK, NHQK, NDEQ, QHRK MILV, MILF, HY, FYW, wherein the single letter amino acid codes are grouped by those amino acids that may be substituted for each other. Likewise, the “weak” group of conserved residues may be any one of the following: CSA, ATV, SAG, STNK, STPA, SGND, SNDEQK, NDEQHK, NEQHRK, VLEVI, HFY, wherein the letters within each group represent the single letter amino acid code.

“Membrane-active peptide variant” or “antimicrobial peptide variant” means a membrane active polypeptide or antimicrobial peptide as defined above or below having at least about 80% amino acid sequence identity with a full-length native membrane-active polypeptide sequence as disclosed herein or as known in the art. Such membrane-active peptide variants include, for instance, membrane-active peptides wherein one or more amino acid residues are added, or deleted, at the N- or C-terminus of the full-length native amino acid sequence. Variants include naturally occurring variants of antimicrobial peptides including those having at least 95% sequence identity to the corresponding naturally occurring peptide and having membrane activity. Ordinarily, a membrane-active peptide variant will have at least about 80% amino acid sequence identity, preferably at least about 81% amino acid sequence identity, more preferably at least about 82% amino acid sequence identity, more preferably at least about 83% amino acid sequence identity, more preferably at least about 84% amino acid sequence identity, more preferably at least about 85% amino acid sequence identity, more preferably at least about 86% amino acid sequence identity, more preferably at least about 87% amino acid sequence identity, more preferably at least about 88% amino acid sequence identity, more preferably at least about 89% amino acid sequence identity, more preferably at least about 90% amino acid sequence identity, more preferably at least about 91% amino acid sequence identity, more preferably at least about 92% amino acid sequence identity, more preferably at least about 93% amino acid sequence identity, more preferably at least about 94% amino acid sequence identity, more preferably at least about 95% amino acid sequence identity, more preferably at least about 96% amino acid sequence identity, more preferably at least about 97% amino acid sequence identity, more preferably at least about 98% amino acid sequence identity and most preferably at least about 99% amino acid sequence identity with a full-length native membrane-active peptide sequence as disclosed herein or known in the art. Ordinarily, membrane-active variant polypeptides are at least about 10 amino acids in length, often at least about 20 amino acids in length, more often at least about 30 amino acids in length, more often at least about 40 amino acids in length, more often at least about 50 amino acids in length, more often at least about 60 amino acids in length, more often at least about 70 amino acids in length, more often at least about 80 amino acids in length, more often at least about 90 amino acids in length, more often at least about 100 amino acids in length, more often at least about 150 amino acids in length, more often at least about 200 amino acids in length, more often at least about 300 amino acids in length, or more.

“Percent (%) amino acid sequence identity” with respect to the membrane-active peptide sequences identified herein or known in the art is defined as the percentage of amino acid residues in a candidate sequence that are identical with the amino acid residues in the specific membrane-active peptide sequence, after aligning the sequences and introducing gaps, if necessary, to achieve the maximum percent sequence identity, and not considering any conservative substitutions as part of the sequence identity. Alignment for purposes of determining percent amino acid sequence identity can be achieved in various ways that are within the skill in the art, for instance, using publicly available computer software such as BLAST, BLAST-2, ALIGN or Megalign (DNASTAR) software. Those skilled in the art can determine appropriate parameters for measuring alignment, including any algorithms needed to achieve maximal alignment over the full length of the sequences being compared.

Percent amino acid sequence identity values may also be obtained as described below by using the WU-BLAST-2 computer program (Altschul et al., Methods in Enzymology 266:460-480 (1996)). Most of the WU-BLAST-2 search parameters are set to the default values. Those not set to default values, i.e., the adjustable parameters, are set with the following values: overlap span=1, overlap fraction=0.125, word threshold (T)=I1, and scoring matrix=BLOSUM62. When WU-BLAST-2 is employed, a % amino acid sequence identity value is determined by dividing (a) the number of matching identical amino acid residues between the amino acid sequence of the membrane-active peptide of interest having a sequence derived from the native membrane-active peptide and the comparison amino acid sequence of interest (i.e., the sequence against which the membrane-active peptide of interest is being compared which may be a membrane-active peptide variant polypeptide) as determined by WU-BLAST-2 by (b) the total number of amino acid residues of the membrane-active peptide of interest. For example, in the statement “a polypeptide comprising an the amino acid sequence A which has or having at least 80% amino acid sequence identity to the amino acid sequence B”, the amino acid sequence A is the comparison amino acid sequence of interest and the amino acid sequence B is the amino acid sequence of the Membrane-active peptide of interest.

Percent amino acid sequence identity may also be determined using the sequence comparison program NCBI-BLAST2 (Altschul et al., Nucleic Acids Res. 25:3389-3402 (1997)). NCBI-BLAST2 uses several search parameters, wherein all of those search parameters are set to default values including, for example, unmask=yes, strand=all, expected occurrences=10, minimum low complexity length=15/5, multi-pass e-value=0.01, constant for multi-pass=25, dropoff for final gapped alignment=25 and scoring matrix=BLOSUM62.

In situations where NCBI-BLAST2 is employed for amino acid sequence comparisons, the % amino acid sequence identity of a given amino acid sequence A to, with, or against a given amino acid sequence B (which can alternatively be phrased as a given amino acid sequence A that has or comprises a certain % amino acid sequence identity to, with, or against a given amino acid sequence B) is calculated as follows:

where X is the number of amino acid residues scored as identical matches by the sequence alignment program NCBI-BLAST2 in that program's alignment of A and B, and where Y is the total number of amino acid residues in B. It will be appreciated that where the length of amino acid sequence A is not equal to the length of amino acid sequence B, the % amino acid sequence identity of A to B will not equal the % amino acid sequence identity of B to A.

In other embodiments, the membrane-active peptide or fusion protein thereof, may contain one or more unnatural amino acids (e.g., unnatural side chains, unnatural chiralities, N-substituted amino acids, or beta amino acids), unnatural topologies (e.g., cyclic or branched) or unnatural chemical derivatives (e.g., methylated or terminally blocked), unnatural backbones including those with partially or totally substituted amide (peptide) bonds with ester, thioester or other linkages.

### Fusion Proteins

Accordingly, one embodiment of the disclosure provides a fusion protein comprising an membrane-active peptide, for example an antimicrobial peptide operably linked to a second peptide. The second peptide can be a heterologous peptide that is not the same as the membrane-active peptide. The second peptide of the fusion protein may be a polypeptide or fragment of a polypeptide of sufficient size, length, or conformation to reduce the effect the antimicrobial peptide-polypeptide fusion protein has on cell permeability compared the antimicrobial peptide alone. Typically, the second peptide has more than about 50 amino acids. Exemplary second peptides include, but are not limited to maltose binding protein, green fluorescence protein (GFP), chloramphenicol acetyltransferase (CAT), thioredoxin (Trx), homologues thereof, or a fragment thereof. The second polypeptide can be selected to increase intracellular permeability of the membrane-active peptide or fusion protein thereof, decrease the pore-forming ability of the membrane-active peptide, or protect the membrane-active peptide from degradation. In other embodiments, the second peptide inhibits, partially or completely, the fusion protein from translocating across a membrane.

Without wishing to be bound by one theory, it is believed that the membrane-active peptides and their corresponding fusion proteins provided herein increase membrane permeability of cell by forming a pore in the membrane or by disrupting the membrane. The pore may be formed by one membrane-active peptide or fusion protein or by a several membrane-active peptides or fusion proteins combining to form a multimeric complex. For example, one membrane-active peptide or fusion protein can produce an α-helical structure to form a pore. Alternatively, the fusion protein can adopt a (S-pleated structure in the membrane and thereby increases membrane permeability. The pore can be of sufficient size to allow small organic molecules or proteins to translocate across the membrane.

In certain embodiments, pores formed by the disclosed membrane-active peptides have an interior diameter of about 1 nm to about 7 nm. It will be appreciated that different membrane-active peptides can produce pores having different interior diameters. To increase membrane permeability to a specific compound, a membrane-active peptide that will produce pores having an interior diameter that will accommodate the compound can be used.

The disclosed membrane-active peptides and fusion proteins can increase permeability of cellular membranes including, but not limited to inner cell membranes and outer cell membranes in the case of Gram-negative bacteria. Increasing the permeability of outer cell membranes can allow substances to enter or leave the cell. For example, small molecules including vitamins, cofactors, amino acids, polypeptides, recombinant polypeptides, nucleic acids, polynucleotides, vectors, intracellular or extracellular reactants, intracellular or extracellular enzymatic reaction products, etc. can enter or leave the cell at increased rates compared to controls. Increased permeability of cell membranes can be achieved using the disclosed compositions and methods without significant cytolysis. For example, cells expressing the disclosed membrane-active peptide or fusion protein thereof continue to grow in culture albeit with a longer doubling time compared to control cells. In certain embodiments, cells expressing the disclosed membrane-active peptide or fusion protein thereof reach log phase during culture.

### Compositions

Still another embodiment provides a composition comprising a membrane permeabilizing agent. The membrane permeabilizing agent may comprise a membrane-active peptide, optionally operably linked to a second peptide, in an amount effective to increase cellular membrane permeability without resulting in cytolysis. The composition can be lyopbilized or can include a physiologically buffered carrier solution. Buffering solutions are known in the art and can buffer pH, osmolarity, etc. to mimic in vivo conditions.

Another embodiment provides a cell comprising one or more of the disclosed membrane-active peptides, one or more of the disclosed membrane-active fusion proteins, or one or more nucleic acids encoding the disclosed membrane-active peptides or fusion proteins. The one or more membrane-active peptides or fusion proteins can produce pores of the same or different sizes in the membrane. Typically, the membrane-active peptide fusion protein comprises a membrane-active peptide operably linked to a second peptide, and increases permeability of an inner cell membrane, outer cell membrane, or combination thereof. In certain aspects, the membrane-active peptide or fusion protein does not result in lysis of the cell. Suitable cells include prokaryotic and eukaryotic cells such as mammalian, gram negative or gram positive bacterial, fungal, or plant cells. Cells expressing on or more of the disclosed membrane-active peptides, fusion proteins, or combination thereof remain viable with increased membrane permeability compared to cells that do not express the disclosed membrane-active peptides or fusion proteins. Typically, the cells expressing the membrane-active peptide or fusion protein have a longer doubling time compared to control cells. Li certain embodiments, the cells can also be engineered to express at least a second recombinant protein, for example a therapeutic polypeptide.

In certain aspects, the one or more of the disclosed fusion proteins increase a cell's membrane permeability to a molecule, protein, or substance that does not have a receptor or natural method for entering the cell.

### Vectors

Another embodiment provides a vector comprising a nucleic acid encoding the disclosed membrane-active peptides and fusion proteins. Suitable vectors include but are not limited to plasmids. The vector optionally contains sufficient control sequences for expressing the nucleic acid in a cell. For example, the vector may include a promoter, typically an inducible promoter. Suitable promoters for expression in E. coli, for example include T7, T5, Lac promoters. Depending on the types of cells used, other promoters could be used including, but not limited to adenoviral promoters, such as the adenoviral major late promoter; or heterologous promoters, such as the cytomegalovirus (CMV) promoter; the respiratory syncytial virus (RSV) promoter; inducible promoters, such as the MMT promoter, the metallothionein promoter; heat shock promoters; the albumin promoter; the ApoAI promoter; human globin promoters; viral thymidine kinase promoters, such as the Herpes Simplex thymidine kinase promoter; retroviral LTRs; the /3-actin promoter; and human growth hormone promoters.

In some embodiments the promoter and positive or negative control elements can be selected to control the amount of expression of the membrane-active polypeptide or fusion protein thereof. For example, when the lac promoter is used, IPTG inducer is added in concentrations effective to achieve a desired level of expression.

### Methods or Use

One embodiment provides a method for increasing the recovery of a recombinant polypeptide from a cell. The method includes expressing in the cell one or more nucleic acids encoding a membrane-active, a fusion protein, or a combination thereof. The fusion comprises a membrane-active peptide operably linked to a second peptide. The membrane-active peptide or corresponding fusion protein increases membrane permeability of the cell, for example by creating a pore in the membrane, and allows the recombinant protein produced by the cell to translocate an inner or outer membrane of the cell without the need of a signal sequence or other proteins. For example, the recombinant polypeptide can translocate from the cytoplasm to the periplasm in a Gram-negative bacterium. The periplasm fraction can be isolated using convention techniques and thereby increasing recovery of the recombinant polypeptide compared to a control cell.

Another embodiment provides a method for increasing the permeability of an outer membrane of cell by contacting the outer membrane with one or more of the disclosed membrane-active peptides, fusion proteins, or combination thereof. The membrane-active peptide or fusion protein can be used in combination with additional permeabilizing agents, including, but not limited to, toxins, surfactants, detergents, organic solvents, or freeze thaw techniques.

Yet another embodiment provides a method for increasing the yield of an enzymatic product from a cell and rate of production. In this method, the cell expresses a nucleic acid encoding one or more of the disclosed membrane-active peptides, fusion proteins, or combination thereof. The membrane-active peptide or fusion protein increases membrane permeability of the cell allowing extracellular reactants to translocate an outer membrane of the cell and contact an enzyme located within the cell. Greater concentrations of reactant can enter the cell at a higher rate compared to a control cell as a result of the increase in outer membrane permeability. Therefore, the enzyme within the cell can produce product at a higher rate, resulting in increased product yield and concentrations.

Another embodiment provides a method for bioremediation. In this method a cell containing one or more enzymes or pathways that convert or is capable of converting a toxic reactant into a non-toxic product is contacted with one or more of the disclosed membrane-active peptides, fusion proteins, combinations thereof, optionally in combination with at least one additional membrane permeabilizing agent. Alternatively, the cell is transfected to express one or more membrane-active peptides, membrane-active fusion proteins, or combinations thereof. The cell is then contacted with the toxic reactant under conditions that favor the conversion of the toxic reactant into a non-toxic product. Typically, the conversion is an enzymatic conversion. The enzyme for converting the toxic reactant can be naturally produced by the cell. Alternatively, the cell can be transfected with a heterologous nucleic acid encoding the enzyme.

Toxic compounds include, but are not limited to those found in insecticides (organophosphates, carbamates, pyrethroids, endosulfan, neonicotinoids, benzoyl ureas), herbicides (glyphosate, paraquat, triazines, phenyl ureas), and fungicides (carbamate). One embodiment provides a method for bioremediation comprising expressing a membrane-active peptide in a cell, wherein the cell also expresses parathion hydrolase or 3-nitrophenol nitroreductase, and contacting the cell with a composition comprising organophosphates such as parathion. Organophosphate hydrolase breaks down toxic organophosphates into less toxic compounds. 3-nitrophenol nitroreductase catalyzes chemoselective reduction of aromatic nitro groups to hydroxylamino groups in the presence of NADPH.

Bacteria that use toxic compounds as sources of nitrogen, sulfur, or carbon are known (Siddiquea, T. et al. (2003) J Environ Qual., January-February; 32(1):47-54). For example, bacteria capable of degrading endosulfan (6,7,8,9,10,10-hexachloro-1,5,5a,6,9,9a-hexahydro-6,9-methano-2,3,4-benzo-dioxathiepin-3-oxide) can be transfected to express a membrane-active peptide or fusion protein thereof which can increase membrane permeability to this cyclodiene organochlorine.

Another embodiment provides a method for controlling membrane permeability of a cell by expressing one or more nucleic acids encoding one or more of the disclosed membrane-active peptides, fusion proteins, or combination thereof. Membrane permeability increases with an increase in number of nucleic acids encoding the membrane-active peptide or fusion protein in the cell. Alternatively, membrane permeability can increase in response to an increase in the amount of inducer for an inducible promoter linked to the nucleic acids. Lastly, promoters of different strengths can be used so that the amount of fusion protein expressed by the cell is controlled. Highly levels of expression of the fusion protein correspond to higher membrane permeability increases compared to low levels of expression of the fusion protein. Alternatively, the cell can be engineered to express a predetermined amount of the disclosed membrane-active peptides or fusion protein and various amounts of a permeabilizing agent, including the disclosed fusion proteins can be added to the cell to increase membrane permeability to a desired level.

It will be appreciated that the membrane permeability can also be regulated by controlling the size of the pores formed by the disclosed fusion proteins. The size of the pore can be tailored to accommodate the size of a desired product, for example a small molecule or protein, so that membrane permeability is selectively increased for a desired product. In situations requiring the delivery of a small organic molecules, for example hydrophobic molecules, to the inside of a cell, the pore size generated by the membrane-active peptide or fusion protein can be smaller than the pore size needed to translocated a recombinant protein across a cell membrane.

Antimicrobial peptides are known in the art and have known or predicted pore sizes. An antimicrobial peptide or membrane-active peptide that forms a pore of a known or defined size in a membrane can be fused with a second peptide, for example a heterologous peptide. This fusion protein can then be expressed in a cell causing the cell to have a greater number of pores having a defined or known size. The cell's permeability to a specific size of molecules is therefore selectively increased.

### Cell-Based Assays for Drug Discovery, Target Validation, Lead Optimization and Biosensor Development

Another embodiment provides a cell-based assay for drug discovery and related research. Drug targets include, but are not limited to one or more proteins expressed in cells, RNA, DNA complexes, small molecule libraries, or protein drugs normally not permeable to the cell membrane could be evaluated using this method. The cell assay can be in the form of a cellular array. The cells used in the array can be transfected with a membrane-active peptide, optionally fused to a second peptide, for example a heterologous peptide, so that the cell has increased permeability to a target compound or reporter compound. The cells of the array can be engineered to produce a detectable phenotypic change in response to the target compound or reporter compound. For example, target compounds can be screened to determine whether they increase activity of a gene of interest. Control sequences specific for the gene of interest can be operably linked to a reporter gene. If the target compound binds to the control sequence, it will cause the reporter gene to become active which in turn causes a detectable phenotypic change in the cell.

In biosensor applications, targets may not be able to get inside cells easily to afford the needed sensitivity. One embodiment provides detecting expression of nucleic acids in a living cell in response to contact with a target compound. In this method, the cell is engineered to express a membrane-active peptide, optionally operably linked to a second peptide. The membrane-active peptide increases membrane permeability of the cell to a reporter nucleic acid, for example a labeled antisense DNA or RNA or molecular beacons. The cells are contacted with one or more target compounds in combination or alternation with the reporter nucleic acid. If the target compound causes an increase in expression of the gene or RNA of interest, the reporter nucleic acid will hybridize with the RNA in the cell and the label will be detected. Indeed, the amount of RNA can be quantitated over different doses of the target compound. This method will allow these targets to be detected, and other targets detected with increased sensitivity.

Another embodiment provides an array comprising units of cells expressing an membrane-active peptide, optionally operably linked to a second polypeptide and deposited at addressable locations of a substrate. For example, each addressable location may contain one or more units of cells or one or more test compounds. The cells may be attached to the array substrate using any conventionally means, for example, polysaccharides, polyamino acids, or a combination thereof.

Another embodiment provides a method including reacting multiple cellular arrays with standard mixtures or additions of test compounds. The method can then include comparing the amount of signal detected at each corresponding location or feature on two or more of the arrays. Standardizing the arrays can be based on this comparison.

Another embodiment provides a method including detecting a first detectable signal (e.g., color) from the disclosed arrays and a second detectable signal from a standard mixture of the control compounds. The method can include comparing the strength of the first and second detectable signals. Quantitating the signal generated by the test compounds with control compounds can be based on this comparison. In the cellular arrays, the cells expressing the disclosed fusion protein can optionally express an enzyme that produces a detectable product when contacted with a specific reagent.

Contacting can include any of a variety of known methods for contacting an array with a reagent, sample, or composition. For example, the method can include placing the array in a container and submersing the array in or covering the array with the reagent, sample, or composition. The method can include placing the array in a container and pouring, pipetting, or otherwise dispensing the reagent, sample, or composition onto features on the array. Alternatively, the method can include dispensing the reagent, sample, or composition onto features of the array, with the array being in or on any suitable rack, surface, or the like.

Detecting can include any of a variety of known methods for detecting a detectable signal from a feature or location of an array. Any of a variety of known, commercially available apparatus designed for detecting signals of or from an array can be employed in the present method. Such an apparatus or method can detect one or more of the detectable labels described herein below. For example, known and commercially available apparatus can detect colorimetric, fluorescent, or like detectable signals of an array. The methods and systems for detecting a signal from a feature or location of an array can be employed for monitoring or scanning the array for any detectable signal. Monitoring or detecting can include viewing (e.g., visual inspection) of the array by a person.

The disclosed arrays or compositions can be provided in any variety of common formats. The cells can be provided in a container including, but not limited to a 96 well microtiter plate or high throughput plate. The cells can be added to the container as a suspension. In an embodiment, each of a plurality of disclosed cells and arrays is provided in its own container (e.g., vial, tube, or well). The present disclosed-cells and arrays or compositions can be provided with materials for creating a cellular array or with a complete cellular array. In fact, the cells can be provided bound to one or more features of a cellular array.

Arrays on a substrate can be designed for testing against any type of sample, whether a trial sample, reference sample, a combination of them, or a known mixture of test compounds. Any given substrate may carry one, two, four or more arrays disposed on a front surface of the substrate. Depending upon the use, any or all of the arrays may be the same or different from one another and each may contain multiple spots or features. A typical array may contain more than ten, more than one hundred, more than one thousand, more than ten thousand features, or even more than one hundred thousand features, in an area of less than 50 cm², 20 cm², or even less than 10 cm², or less than 1 cm². For example, features may have widths (that is, diameter, for a round spot) in the range from a 10 μm to 1.0 cm. In other embodiments each feature may have a width in the range of 1.0 μm to 1.0 mm, of 5.0 μm to 500 μm, or of 10 μm to 200 μm. Non-round features may have area ranges equivalent to that of circular features with the foregoing width (diameter) ranges. Feature sizes can be adjusted as desired, for example by using one or a desired number of pulses from a pulse jet to provide the desired final spot size.

Substrates of the arrays can be any solid support, a colloid, gel or suspension. Exemplary solid supports include, but are not limited to metal, metal alloys, glass, natural polymers, non-natural polymers, plastic, elastomers, thermoplastics, pins, beads, fibers, membranes, or combinations thereof.

At least some, or all, of the features are of different compositions (for example, when any repeats of each feature composition are excluded the remaining features may account for at least 5%, 10%, or 20% of the total number of features), each feature typically being of a homogeneous composition within the feature. Thus, certain features may contain one type of cell as described and a second feature may contain a second type of cell as described. Interfeature areas will typically (but not essentially) be present which do not carry any polynucleotide (or other biopolymer or chemical moiety of a type of which the features are composed). Such interfeature areas typically will be present where the arrays are formed by processes involving drop deposition of reagents but may not be present when, for example, photolithographic array fabrication processes are used. It will be appreciated though, that the interfeature areas, when present, could be of various sizes and configurations.

Array features will generally be arranged in a regular pattern (for example, rows and columns). However other arrangements of the features can be used where the user has, or is provided with, some means (for example, through an array identifier on the array substrate) of being able to ascertain at least information on the array layout (for example, any one or more of feature composition, location, size, performance characteristics in terms of significance in variations of binding patterns with different samples, or the like). Each array feature is generally of a homogeneous composition.

Each array may cover an area of less than 100 cm², or even less than 50 cm², 10 cm², or 1 cm². In many embodiments, the substrate carrying the one or more arrays will be shaped generally as a rectangular solid (although other shapes are possible), having a length of more than 4 mm and less than 1 m, for example, more than 4 mm and less than 600 mm, less than 400 mm, or less than 100 mm; a width of more than 4 mm and less than 1 ni, for example, less than 500 mm, less than 400 mm, less than 100 mm, or 50 mm; and a thickness of more than 0.01 mm and less than 5.0 mm, for example, more than 0.1 mm and less than 2 mm, or more than 0.2 and less than 1 mm. With arrays that are read by detecting fluorescence, the substrate may be of a material that emits low fluorescence upon illumination with the excitation light. Additionally in this situation, the substrate may be relatively transparent to reduce the absorption of the incident illuminating laser light and subsequent heating if the focused laser beam travels too slowly over a region. For example, the substrate may transmit at least 20%, or 50% (or even at least 70%, 90%, or 95%), of the illuminating light incident on the front as may be measured across the entire integrated spectrum of such illuminating light or alternatively at 532 nm or 633 nm.

Arrays can be fabricated using drop deposition from pulse jets of either test compound solutions or units of encapsulated cells. Other drop deposition methods can also be used for fabrication.

### Methods Employing Arrays

Following receipt by a user of an array made according to the present disclosure, it will typically be exposed to a sample (for example, a test compound) in any well known manner and the array is then read. Reading of the array may be accomplished by illuminating the array and reading the location and intensity of resulting fluorescence at multiple regions on each feature of the array. Arrays may be read by any method or apparatus known in the art, with other reading methods including other optical techniques (for example, detecting chemiluminescent or electroluminescent labels) or electrical techniques (where each feature is provided with an electrode to detect hybridization at that feature). Data from read arrays may be processed in any known manner, such as from commercially available array feature extraction software packages. A result obtained from the reading followed by a method of the present invention may be used in that form or may be further processed to generate a result such as that obtained by forming conclusions based on the pattern read from the array (such as whether or not a particular target sequence may have been present in the sample, or whether or not a pattern indicates a particular condition of an organism from which the sample came). A result of the reading (whether further processed or not) may be forwarded (such as by communication) to a remote location if desired, and received there for further use (such as further processing).

Detectable Labels

The disclosed cells and arrays can include a detectable label, for example, a first detectable label. A second detectable label can be generated when the test compound contacts the cells on an array. Suitable labels include radioactive labels and non-radioactive labels, directly detectable and indirectly detectable labels, and the like. Directly detectable labels provide a directly detectable signal without interaction with one or more additional chemical agents. Suitable of directly detectable labels include colorimetric labels, fluorescent labels, and the like. Indirectly detectable labels interact with one or more additional members to provide a detectable signal. Suitable indirect labels include a ligand for a labeled antibody and the like.

Suitable fluorescent labels include: xanthene dyes, e.g., fluorescein and rhodamine dyes, such as fluorescein isothiocyanate (FITC), 6-carboxyfluorescein (commonly known by the abbreviations FAM and F), 6-carboxy-2′,4′,7′,4,7-hexachlorofluorescein (HEX), 6-carboxy-4′,5′-dichloro-2′,7′-dimethoxyfluorescem (JOE or J), N,N,N′,N′-tetramethyl-6-carboxyrhodamine (TAMRA or T), 6-carboxy-X-rhodamine (ROX or R),5-carboxyrhodamine-6G (R605 or G5), 6-carboxyrhodamine-6G (R6G6 or G6), and rhodamine 110; Alexa dyes, e.g. Alexa-fluor-547; cyanine dyes, e.g., Cy3, Cy5 and Cy7 dyes; coumarins, e.g., umbelliferone; benzimide dyes, e.g., Hoechst 33258; phenanthridine dyes, e.g., Texas Red; ethidium dyes; acridine dyes; carbazole dyes; phenoxazine dyes; porphyrin dyes; polymethine dyes, e.g., cyanine dyes such as Cy3, Cy5, etc; BODEPY dyes and quinoline dyes.

### Kits

Another embodiment provides a kit comprising one or more of the disclosed membrane-active peptides or fusion proteins thereof, one or more nucleic acids encoding one or more of the disclosed membrane-active peptides or fusion proteins, or a combination thereof. The kit optionally includes a buffered carrier solution to buffer the pH and/or salt concentration. At least one additional membrane permeabilizing agent may be included with the kit. Another embodiment provides kit including a cell expressing one or more of the disclosed membrane-active peptides or fusion proteins. Buffered solutions and cell culture media may also be included in the kit.

It should be noted that, as used in this specification and the appended claims, the singular forms “a,” “an,” and “the” include plural referents unless the content clearly dictates otherwise. Thus, for example, reference to a composition containing “a compound” includes a mixture of two or more compounds. It should also be noted that the term “or” is generally employed in its sense including “and/or” unless the content clearly dictates otherwise.

### EXAMPLES

### Materials and Methods

The following exemplary methods and materials were used to provide the data presented in the accompanying working examples.

### Materials

N-phenyl-1-naρhthylamine (NPN), o-nitrophenyl-β-D-galactoside (ONPG),j!?-nitrophenyl-β-D-glucuronide were purchased from Sigma Chemical Co. (St. Louis, Mo.), Isopropyl-β-D-thiogalactopyranoside (IPTG) and restriction enzymes were from Promega (Madison, Wis.). Nitrocefin was procured from BD Biosciences (San Jose, Calif.).

### Construction MalE-MagllFusion Protein

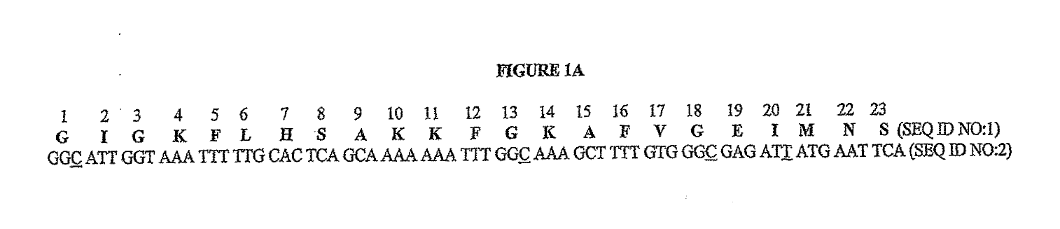

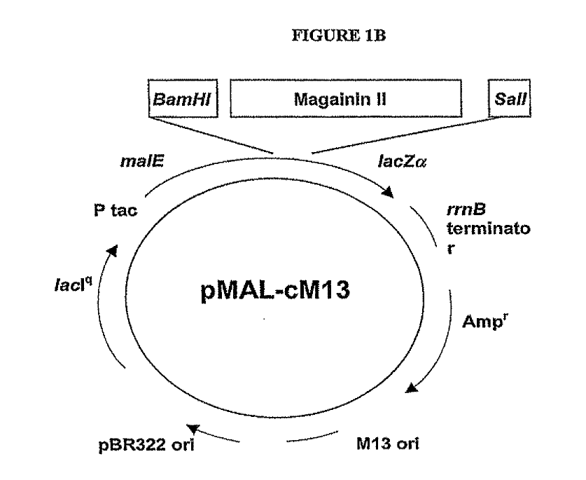

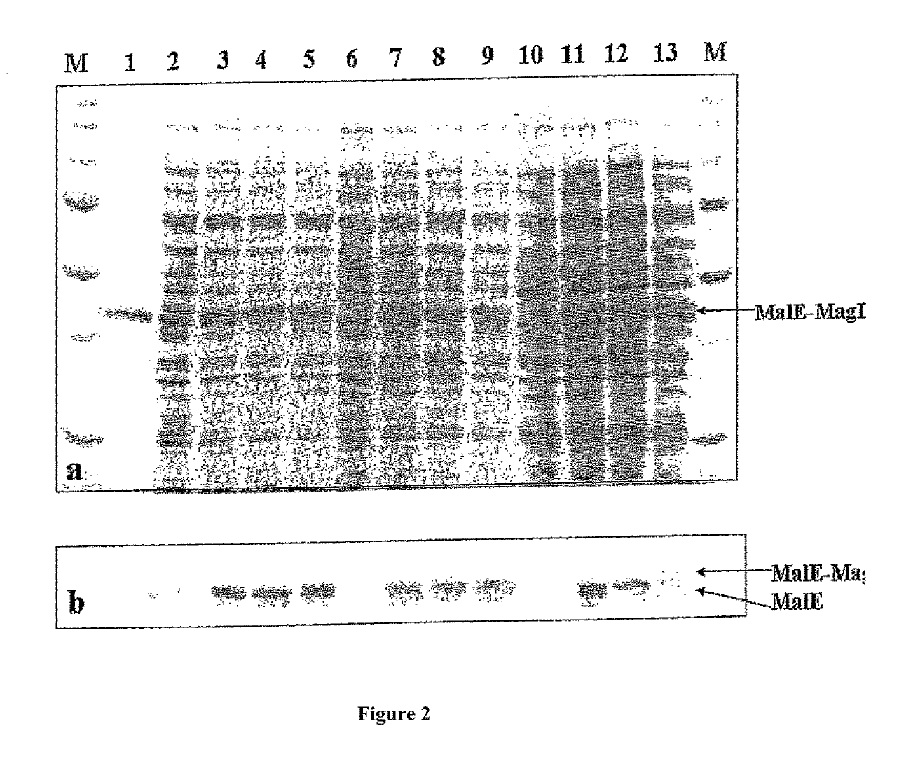

The pMAL fusion expression system (New England Biolab, Beverly, Mass.) was used to clone and express the Magainin II peptide as a fusion to the maltose binding protein (MaIE). In particular, Escherichia coli TB1 was used as the host strain, and the cytoplasmic expression vector pMAL-c2x was used to construct the fusion expression vector (FIG. IB and Table 2). The gene encoding Magainin II from Xenopus laevis (GenBank Accession #J03193) was optimized according to the E. coli codon usage (SEQ ID NO:2), and synthesized by Invitrogen (Carlsbad, Calif.) flanked with restriction sites BamAl and SaR to facilitate the directional cloning into pMAL-c2x (FIG. 2). Briefly, the two single strands, 5′-GATCCTGGGG CATTGGTAAA TTTTTGCACT CAGCAAAAAA ATTTGGCAAA GCTTTTGTGG GCGAGATTAT GAATTCATAA TAG-3′ (SEQ ID NO: 12) and 5′-TCGA CTATTATGAA TTCATAATCT CGCCCACAAA AGCTTTGCCA AATTTTTTTG CTGAGTGCAA AAATTTACCA ATGCCCCAG-3′ (SEQ ID NO: 13) were annealed, cut with HindRI, which is located in the middle of the Magainin II gene, and examined on an agarose gel. Once successful annealing was confirmed, the DNA fragment was ligated into pMAL-c2x digested with Bω\dot{r}AI and Sail. The transformation was carried out by electroporation using a MicroPulser (Biorad, Hercules, Calif.), and the white colonies were randomly picked from LB/Amp/X-gal/IPTG plates. The recombinants were subjected to DNA sequencing using primer 5′-GGTCGTCAGACTGTCGATGAAGCC-S′ (SEQ ID NO: 14) at the DNA Sequencing Core Lab of Georgia Institute of Technology. Sequences were analyzed using DNAMAN software (Lynnon Corp., Quebec, Canada). The positive clone cM13 was confirmed with the correct DNA sequence and in frame with MaIE. The plasmid was named pMal-cM13 and was transformed into E. coli E609 and E609L for subsequent study.

### The Expression of MalE-Magll and Effect on Growth

Single colonies of the E. coli E609 strain carrying either pMAL-c2x or pMAL-cM13 were inoculated into 5 ml of LB/Amp¹⁰⁰ and grown overnight at 37° C. The overnight cultures were then inoculated into 10 ml of LB/Amp¹⁰⁰ broth (2%) in the absence or presence of isopropyl-β-D-thiogalactopyranoside (IPTG) at desired concentrations (0, 0.1, 0.3, and 0.5 mM). Samples were taken hourly for OD₆ₒₒ measurement with a UV/Vis spectrophotometer (Beckman DU530, Beckman Coulter, Fullerton, Calif.).

For protein expression analysis, one milliliter culture was withdrawn at 3, 4, and 5 hrs. Cell pellets were collected by centrifugation at 13,000×g for 2 min. Cell pellets were then re-suspended in 50 μl Laemmli Sample Buffer with 2-Mercaptoethanol, boiled for 5 min. The supernatant was collected by centrifugation. The 10 μl sample was loaded onto a SDS-PAGE gel for protein expression analysis. The western blot analysis was then carried out with a WesternBreeze® Chromogenic Western Blot Immunodetection kit (Invitrogen, Carlsbad, Calif.) on a Nitrocellulose membrane (BioRad, Hercules, Calif.), following the manufacturer's instruction.

### Cellpreparation for Permeability Assay

E. coli E609 cells carrying either pMAL-c2x or pMAL-cM13 fusion constructs (Table 2) were harvested in the mid-log-phase (growth conditions as above) by centrifugation at 4,000×g for 10 min, washed once, and then resuspended in either 10 mM HEPES (pH 7.3) or 50 mM Sodium phosphate buffer (pH 7.4) to 1.0 A₆₀₀.

TABLE 2
Bacterial strains and plasmids

| Strains or | | |
|---|---|---|
| Plasmids | Description | Source or reference |
| Strains | | |
| E. coli K12 TB1 | F am A(lac-proAB) [Φ80dlac | New England Biolabs |
| | A(lacZ)M15] rpsL(Sti^{R}) thi fisdR | |
| E. coli B609L | /p/?::Tn/0; periplamic leaky; Tc ^{r} | Yem and Wu, 1978 |
| E. coil E609 | HfrCpps, isogenic parent of | Yem and Wu, 1978 |
| | E609L | |
| Plasmids | | |
| c2x | cytoplasmic expression | New England Biolabs |
| | vector fused with | |
| | maltose binding protein (MaIE) | |
| cM13 | cytoplamsic expression of | This study |
| | Magainin 11 fused with MaIE | |

### Preparation of Cell-Free Extracts

Cells were harvested by centrifugation, resuspended with 10 mM HEPES or sodium phosphate buffer containing 5 mM dithiothreitol (DTT), then subjected to sonication using Sonifier* Cell Disrupters (Branson Ultrasonics, Danbury, Conn.) at 15 sec×4 bursts with 45 sec intervals. The supernatant was collected by centrifugation at 13,000×g for 10 min at 4° C.

### Cellular Location ø/MagII Fusion

To determine the cellular location of cytoplasmic expressed MagII fusion, the periplasmic fractionation of induced E609/cM13 was prepared using lysozyme and osmotic shock treatments (Ni and Chen, 2004). Cell pellets from 40 ml of culture broth were resuspended in 2 ml OSI (200 mM Tris-HCl (pH 7.8), 2.5 mM EDTA, 2 mM CaCl₂ and 20% sucrose) with 100 μg/ml lysozyme, and incubated at room temperature for 15 min. After addition of 2 ml ice-cold water, the suspension was incubated for another 15 min The supernatant was collected by centrifugation at 13,000×g for 15 min at 4° C. followed by concentration using an Ultrafree®-CL microcentrifuge filter with 10 kDa NMWL (Millipore, Bedford, Mass.), then analyzed with SDS-PAGE and Western blot.

### NPN Outer Membrane Permeability Assay

E. coli cells were grown as described above, harvested by centrifugation at room temperature, and resuspended with 10 mM HEPES buffer to ODeoo of 1.0. Assay was modified to that previously described (Helander and Mattila-Sandholm, 2000). 100 μl of a N-ρhenyl-1-na ρhthylamine (NPN) stock solution (20 μM) was first added in a black 96-well assay plate with a clear bottom (Costar®363 1, Corning Incorporated, Corning, N.Y.). 100 μl of cell suspension or HEPES buffer as control was pipetted into the wells immediately before the measurement. The fluorescence was monitored using a Victor III microplate reader (Perkin Elmer, Boston, Mass.) with excitation and emission wavelengths set to 350 and 420 nm, respectively. The NPN uptake factor was calculated as a ratio of background-corrected (subtracted by the value in the absence of NPN) fluorescence values of the cell suspension to that of the HEPES buffer. All data presented were averages of at least three separate experiments.

### β-Lactamase Assay

The β-lactamase assay was carried out in 96-well microtiter plates with 200 μl total volume per well containing 20 μg/ml nitrocefm and cells (0.1 A₆Oo), or an appropriate volume of an extracellular fraction, in HEPES buffer. The initial velocities of nitrocefm cleavage (e_{5O}o=15,OOO M⁻¹Cm′¹) were followed by absorption at 490 nm using a Victor III microplate reader (Perkin Elmer, Boston, Mass.). One unit of β-lactamase was defined as the amount of enzyme required to hydrolyze 1 μmol of nitrocefin per minute at 25° C.

### ONPG Inner Membrane Permeability Assay

The inner membrane permeability was evaluated by the entry to the cytoplasm of o-nitrophenyl-β-D-galactoside (ONPG), the substrate for the intracellular enzyme β-galactosidase (Liao et al., 2004). The substrate cleavage was monitored using a Victor III microplate reader at 405 nm. A one-milliliter sample was taken every hour during exponential phase. Pellets were collected by centrifugation at 13,000×g for 2 min and then resuspended in 1 ml HEPES buffer. The reaction was started by adding 100 μl of ONPG (5 mg/ml) to the cell suspension. After incubating for 10 min at 30° C., the reaction was stopped by adding 0.4 ml of 1 M sodium carbonate. The absorption at 405 nm from the reaction product in the supernatant (collected after removal of cell pellets) was measured. The β-galactosidase activity in the cell free extract from the same amount of cells was also determined, and was used as an enzyme activity reference without the impedance of cell membranes.

### Inner Membrane Permeability β-Glucuronidase Assay

Another intracellular enzyme, β-glucuronidase, was used to further evaluate the alteration of the inner membrane permeability. After cells were harvested and washed with a 50 mM sodium phosphate buffer (as described above), 100 μl of cell suspension was mixed with 2 mM DTT, 1 mM p-nitrophenyl-β-D-glucuronide in a well with a final volume of 200 μl. The change of absorbance at 405 nm was monitored at 37° C. using a Victor III microplate reader. One unit of β-glucuronidase activity is the amount of enzyme that liberates 1 nmol of p-nitrophenol per min. A molar extinction coefficient for j?-nitrophenol of 17,700 M^{-1.}cm⁻¹ was used. Total β-glucuronidase activity was determined on cell free extracts after freeze-thaw and sonication (Novel et ah, 1974).

### Example 1

### MagII Cloning and Expression

The codon-optimized gene corresponding to the pore-forming peptide Mag II was synthesized along with the two restriction sites BamBl and Sail, and cloned into a cytoplasmic expression vector, c2x, as a fusion to the maltose binding protein (MaIE) (FIG. IB). The fusion strategy was chosen as short peptides are particularly susceptible to proteases. The expression is under the control of the Tac promoter, inducible with IPTG. One clone, referred as cM13, was one of many clones obtained that had the correct sequence and was in frame with MaIE, and was chosen for further study.

The successful expression of MagII in E. coli was evident from the SDS-PAGE analysis (FIG. 2A). As shown in FIG. 2A, the fusion protein appeared as a strong band with the correct size. The expression apparently was dependent on the inducer concentration and increased with time. However, there was only a limited increase in expression at IPTG concentrations greater than 0.3 mM, and at time points beyond four hours after the induction. The Western blot analysis using the antibody against MaIE showed two bands in all induced samples but not in un-induced ones, a strong band corresponding to the molecular size of the MalE-Magll fusion and a weak band corresponding to MaIE, suggesting that a small fraction of the fusion was degraded (FIG. 2B). But a majority of the fusion was intact and the degraded fraction did not seem to significantly increase with the time and with the inducer concentration.

### Example 2

### MagII Expression on Growth

The effect of MagII expression on cell growth was investigated under different inducer concentrations. As shown in Table 3, at low inducer concentration (0.1 mM), the cells expressing MagII (E609/cM13) exhibited almost identical growth rate as the control (E609/c2x) with a similar doubling time (1.04 hr vs. 0.98 hr), and the final OD reached by the cells expressing MagII was 10% lower than that of the control. Together, these data suggest that there were no significant adverse growth effects when magainin expression was low. But as IPTG concentration increased to 0.3 mM, the magainin expression exerted a negative effect on cell growth, increasing its doubling time significantly (1.52 hr. vs. 0.96 hr). However, despite the significant reduction in growth rat; the final OD was only reduced by about 12%. Further increase of IPTG concentration to 0.5 mM did not seem to reduce the growth rate further and the final OD reached was similar. This can be explained by the observation that no significant increase of MagII expression at higher IPTG concentrations as compared to that at 0.3 mM (FIG. 2 A&B).

TABLE 3

| | | | 0.1 mM | 0.3 mM | 0.5 mM |
|---|---|---|---|---|---|
| Growth indicator | Strain | Control | IPTG | IPTG | IPTG |
| Doubling time | E609/c2x | 0.79 ± 0.05 | 0.98 ± 0.09 | 0.96 ± 0.05 | 1.04 ± 0.01 |
| | E609/cM13 | 0.76 ± 0.04 | 1.04 ± 0.08 | 1.52 ± 0.21 | 1.59 ± 0.25 |
| Final OD₆₀₀ | E609/c2x | 2.63 ± 0.28 | 2.24 ± 0.23 | 2.12 ± 0.20 | 2.11 ± 0.04 |
| | E609/cM13 | 2.53 ± 0.17 | 2.01 ± 0.19 | 1.87 ± 0.22 | 1.90 ± 0.39 |

### Example 3

### Magainin II Expression Increases the Permeability of the Outer Membrane

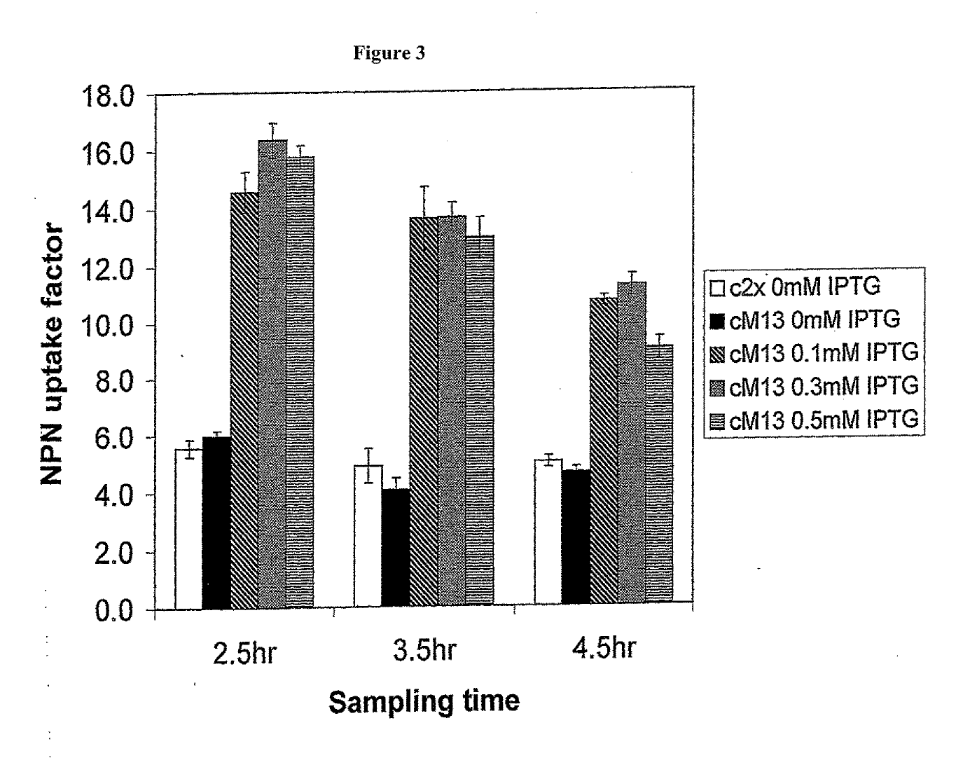

The alteration of the outer membrane permeability due to MagII expression was analyzed using a fluorescent probe, 1-N-phenylnaphthylamine (NPN). The uptake of NPN is normally blocked by an intact outer membrane. It fluoresces weakly in an aqueous environment; but once it has penetrated the outer membrane through a permeablizing mechanism, it gives a strong signal in a hydrophobic membrane (phospholipid) environment. This property is often exploited to detect the integrity of the outer membrane and measure its permeability (Helander and Mattila-Sandholm, 2000). Upon addition of NPN, the cells expressing MagII gave much stronger fluorescent signals than those not expressing the peptide. Subtracting the background reading, the NPN uptake factor was calculated as a basis for a quantitative comparison (Table 4). The uptake factor of NPN for cells expressing the peptide was about 4 times higher than the control, indicating a significant change in outer membrane permeability (FIG. 2). The uptake factor did not seem to change significantly with the IPTG concentrations tested (0.1-0.5 mM). The NPN uptake factor at different time points after induction was also measured. As shown in FIG. 3, the uptake factor decreased slightly with the sampling time, suggesting that the effect of MagII diminished with time. This is not due to cell lysis, as the cells were in the mid-log-phase when these samples were taken.

TABLE 4
The N-phenyl-1-naphthylamine (NPN) uptake assay with E609/c2x and
E609/cM13 induced with different IPTG concentrations (Samples were
taken at 3.5 hr after inoculation and induction).

| | | | Fluorescence | | Fold |
|---|---|---|---|---|---|
| | | Fluorescence | value after | NPN | change |
| | | value | background | uptake | (cm13/ |
| Sample | NPN | (Mean ± SD) | subtraction | factor | c2x) |
| HEPES buffer | − | 951 ± 41 | 647 | 1 | |
| | + | 1598 ± 53 | | | |
| C2x w/o IPTG | − | 679 ± 41 | 3174 | 4.9 | |
| | + | 3853 ± 413 | | | |
| C2x w/ 0.1 mM | − | 708 ± 51 | 2055 | 3.2 | |
| IPTG | + | 2763 ± 291 | | | |
| C2x w/ 0.3 mM | − | 755 ± 7 | 2128 | 3.3 | |
| IPTG | + | 2882 ± 227 | | | |
| C2x w/ 0.5 mM | − | 704 ± 30 | 2116 | 3.3 | |
| IPTG | + | 2820 ± 403 | | | |
| CM13 w/o IPTG | − | 774 ± 69 | 2632 | 4.1 | 0.8 |
| | + | 3405 ± 200 | | | |
| CM13 w/0.1 mM | − | 755 ± 55 | 8806 | 13.6 | 4.3 |
| IPTG | + | 9562 ± 698 | | | |
| CM13 w/ 0.3 mM | − | 773 ± 18 | 8837 | 13.7 | 4.2 |
| IPTG | + | 9610 ± 326 | | | |
| CM13 w/ 0.5 mM | − | 764 ± 26 | 8373 | 12.9 | 4.0 |
| IPTG | + | 9137 ± 411 | | | |

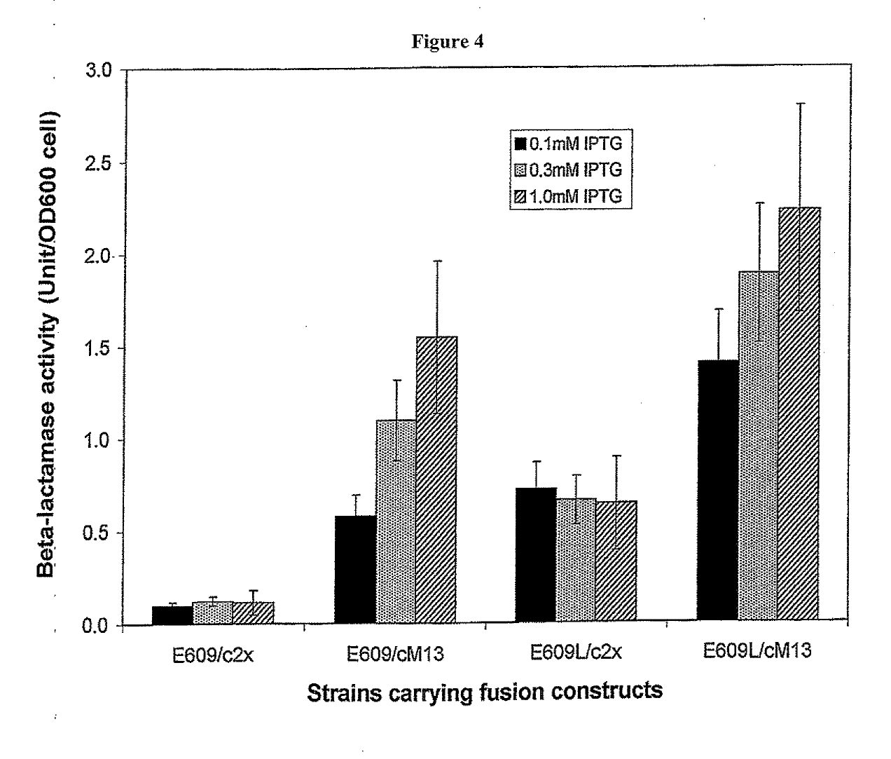

It was observed that the MagII expression caused a significant leakage of periplasmic β-lactamase. As a result, a significant portion of the enzyme activity was found in the supernatant (53% for E609/cM 13 vs. 5% for E609/c2x at 1 mM IPTG). More leakage occurred when the inducer concentration was higher (FIG. 4). The amount of leakage also depended on the genetic background of the host cells. In the lipoprotein mutant E609L, which has a defective Braun's lipoprotein, the expression of Mag II resulted in a much greater leakage (68% for E609L/cM13 at 1 mM IPTG). This result suggests that the expression of the pore-forming peptide altered the outer membrane structure to the extent that allowed proteins to translocate to the growth medium. The expression of such pore-forming peptides, along with an appropriate choice of host strains, could be developed as a method for recovery of periplasmic-expressed proteins.

### Example 4

### Magainin Expression Increases Whole-Cell Activities of Intracellular Enzymes

As MagII was synthesized intracellularly, it must transverse the inner membrane to exert an effect on the outer membrane permeability, implying that its expression also affects the inner membrane integrity and permeability. This was evident from the activity measurement of an intracellular enzyme, β-galactosidase. Whole-cell activities of cells expressing the peptide under different inducer concentrations were compared to their respective controls (Table 5). Cells with the peptides exhibited up to 2.1 fold higher activity than those without. The increase of whole-cell activity of this intracellular enzyme correlated with the inducer concentrations.

TABLE 5
The β-galactosidase activity assay with cell
extracts and whole-cells(E609/c2x and
E609/cM13) induced with different IPTG concentrations

| | A405/OD₆₀₀ cell | | | |
|---|---|---|---|---|
| | | | Percentage | |
| | | | activity | Fold |
| | | Cell free | (whole cell/ | change |
| | Whole cell | extract | cell free | (cM13/ |
| Sample | (Mean ± SD) | (Mean ± SD) | extract %) | c2x) |
| C2x w/0.1 mM | 0.25 ± 0.04 | 3.21 ± 0.42 | 7.7 | |
| IPTG | | | | |
| C2x w/0.3 mM | 0.32 ± 0.06 | 4.03 ± 0.76 | 7.9 | |
| IPTG | | | | |
| C2x w/0.5 mM | 0.32 ± 0.04 | 4.49 ± 0.78 | 7.1 | |
| IPTG | | | | |
| CM13 w/0.1 mM | 0.43 ± 0.06 | 3.46 ± 1.33 | 12.4 | 1.6 |
| IPTG | | | | |
| CM13 w/0.3 mM | 0.60 ± 0.03 | 4.09 ± 1.08 | 14.6 | 1.9 |
| IPTG | | | | |
| CM13 w/0.5 mM | 0.59 ± 0.03 | 3.90 ± 0.4 | 15.1 | 2.1 |
| IPTG | | | | |

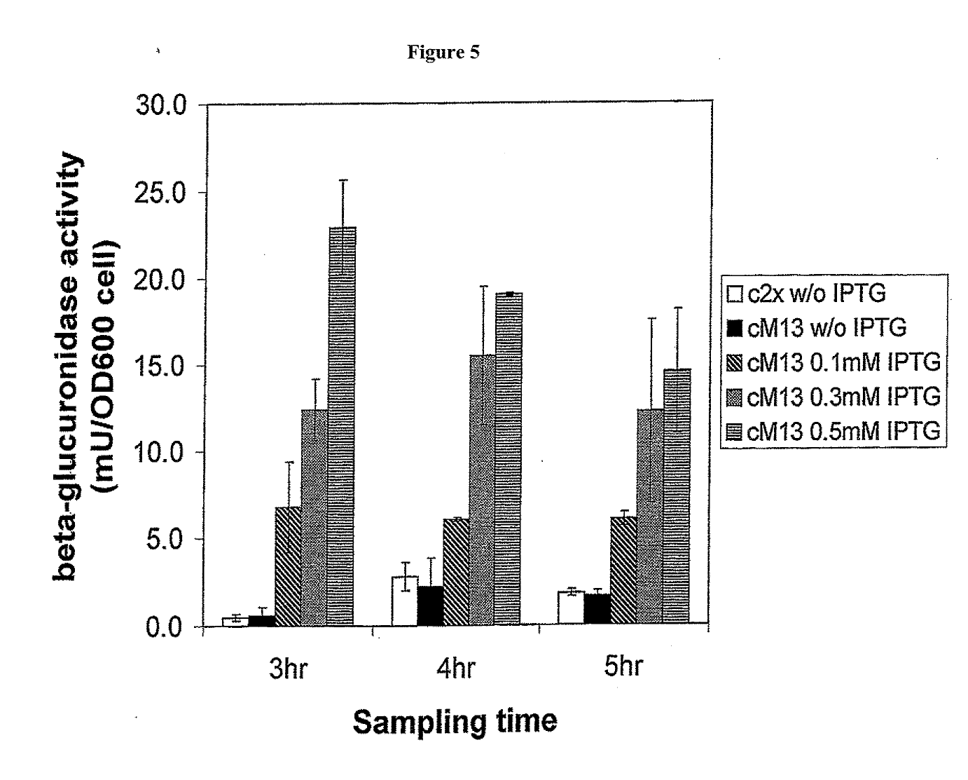

The possible alteration of inner membrane permeability was also probed with another molecule, />>-nitrophenyl-β-D-glucuronide, the substrate of an intracellular enzyme, β-glucuronidase. This enzyme is commonly used as an indicator of coliforms contamination of water supplies and recreational water (Tryland and Fiksdal, 1998). The test is usually done with whole-cells. The access of the substrate by the enzyme apparently was an issue since the whole-cell activities was very low compared to the cell-free extract (Table 6). Less than 3% of the total activity was measured in whole-cells. Expressing MagII increased the activity significantly (FIG. 4). The permeabilizing effect of MagII was dependent upon the inducer concentration, indicating that it could be effectively used to modulate the permeability according to the needs of bioprocesses. At high MagII expression (0.5 niM IPTG concentration), an over 35 fold increase in whole-cell activity was observed, raising the percentage of whole-cell activity to 42% of the cell extract level (Table 6). This indicates that expressing a pore-forming peptide is an effective strategy to increase substrate permeability thereby whole-cell catalyzed reactions. Interestingly, the MagII effect on whole-cell activities showed a time-dependent decline (FIG. 5). This is reminiscent of the NPN data, suggesting that repair mechanisms kicked in to counter the peptide permeabilizing effect. Further studies are needed to elucidate the mechanisms and the process of repairing.

TABLE 6
The β-glucuronidase assay using p-
nitrophcnyl-β-D-glucuronide
cell extracts and whole-cells (E609/c2x and
E609/cM13) induced with different
IPTG concentrations (Samples were taken at 3 hr after
inoculation and induction).

| | mU/OD₆₀₀ cell | | |
|---|---|---|---|
| | | | Percentage |
| | | | activity |
| | | Cell free | (whole cell/ |
| | Whole cell | extract | cell free extract |
| Sample | (Mean ± SD) | (Mean ± SD) | %) |
| C2x w/o IPTG | 0.49 ± 0.28 | 55.56 ± 8.60 | 0.9 |
| C2x w/0.1 mM IPTG | 0.19 ± 0.13 | 31.56 ± 5.58 | 0.6 |
| C2x w/0.3 mM IPTG | 0.56 ± 0.34 | 32.89 ± 2.89 | 1.7 |
| C2x w/0.5 mM IPTG | 0.94 ± 0.34 | 36.45 ± 3.43 | 2.6 |
| CM13 w/0 mM IPTG | 0.59 ± 0.45 | 50.42 ± 8.20 | 1.2 |
| CM13 w/0.1 mM | 6.78 ± 2.06 | 42.35 ± 5.18 | 16.0 |
| IPTG | | | |
| CM13 w/0.3 mM | 12.39 ± 2.26 | 48.70 ± 8.49 | 26.5 |
| IPTG | | | |
| CM13 w/0.5 mM | 22.94 ± 3.67 | 54.19 ± 10.44 | 42.3 |
| IPTG | | | |

### Example 5

### Translocation of MagII Fusion

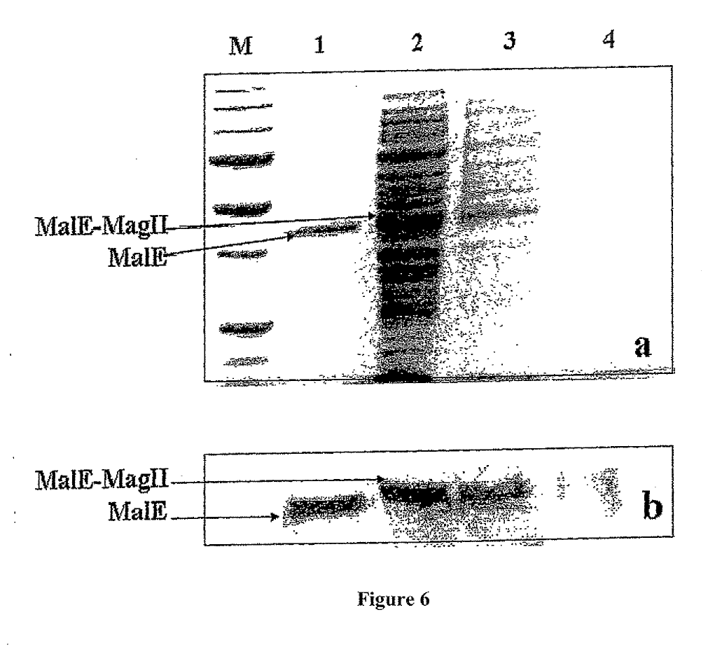

The observed permeability changes in the inner and outer membrane suggest that MagII fusion was able to transverse the cytoplasmic membrane. This would mean that a cytoplasmic protein could be translocated to the periplasmic space without a signal peptide and without the assistance of other proteins. We sought evidence for the presence of the fusion in the periplasmic space. The periplasmic space was fractionated and concentrated by approximately 10 fold and the samples were analyzed using SDS-PAGE and Western blot. The presence of the MagII was evident from the Western blot analysis (FIGS. 6A&B), indicating that the membrane activity of the MagII peptide was indeed sufficient in bringing the fusion protein from one cellular compartment to another. This could work with other proteins. Therefore, fusing a membrane-active peptide provides a novel method to direct a protein of choice to a desired cellular location. This self-promoted, signal-peptide independent mechanism of protein translocation might be useful in facilitating recombinant protein production, protein drug-screening, in cell-based assays, and whole-cell biocatalysis to circumvent the inner membrane permeability issues.

All of the compositions and methods disclosed and claimed herein can be made and executed without undue experimentation in light of the present disclosure. While the compositions and methods of this invention have been described in terms of preferred embodiments, it will be apparent to those of skill in the art that variations may be applied to the composition, methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims. Accordingly, the exclusive rights sought to be patented are as described in the claims below.

1-32. (canceled)

33. A method for increasing the recovery of a recombinant polypeptide from a cell comprising: expressing in the cell a nucleic acid encoding membrane-active peptide or fusion protein thereof according to claim 1, wherein the membrane-active peptide or fusion protein thereof increases membrane permeability of the cell and allows the recombinant protein produced by the cell to translocate one or more cellular membranes thereby increasing recovery of the recombinant polypeptide compared to a control cell.

34. A method for increasing the yield of an enzymatic product from a cell comprising: expressing in the cell a nucleic acid encoding a membrane-active peptide or fusion protein thereof according to claim 1, wherein the membrane-active peptide or fusion protein thereof increases membrane permeability of the cell allowing extracellular reactants to translocate cellular membranes of the cell at a higher rate and thereby the conversion rate of the reactants to products is much higher compared to a control cell.

35. A method for bioremediation comprising: (a) contacting a cell comprising an enzyme capable of converting a toxic reactant into a non-toxic product with the membrane-active peptide or fusion protein thereof of claim 1; and (b) contacting the cell with the toxic reactant under conditions that favor the conversion of the toxic reactant into a non-toxic product, wherein the toxic reactant is converted into the non-toxic product.

36. A cellular array comprising: a plurality of cells according to claim 18 positioned at addressable locations on a solid support, wherein the plurality of cells produce a detectable phenotypic change in the presence of a reactant.

37. The cellular array according to claim 36, wherein the detectable phenotypic change is selected from the group consisting of a change in color, shape, number of cells, apoptosis, or production or a detectable label.

38. A method for controlling membrane permeability of a cell, comprising: expressing one or more vectors of claim 14 in the cell, wherein membrane permeability increases with an increase in number of vectors in the cell, an increase in amount of inducer in contact with the cell, or an increase in strength of the promoter of the one or more vectors or other genetic elements within the vector.
